(19) United States
(12) Patent Application Publication
(10) Pub. No.: US 2012/0088841 A1
(43) Pub. Date: Apr. 12, 2012

(54) COMPOSITION AND A METHOD OF TREATING CNS DISORDERS AND HYPERPIGMENTATION

(76) Inventors: Muhammed Majeed, East Windsor, NJ (US); Sarang Bani, Bangalore (IN); Anjali Pandey, Bangalore (IN); Susmitha Anand-Tathapudi, Bangalore (IN)

(21) Appl. No.: 12/900,727

(22) Filed: Oct. 8, 2010

(51) Int. Cl. A61K 31/05; A61P 25/28; C12N 5/071; A61K 8/34; A61Q 19/02; A61P 25/00; A61P 25/16

(52) U.S. Cl. 514/733; 435/375

(57) ABSTRACT

The present invention relates to a method of treating CNS disorders, particularly Alzheimer's disease. The method comprise step of administering to a subject in need thereof a therapeutically effective amount of hydroxychavicol and/or its derivatives or a composition comprising hydroxychavicol and/or its derivatives optionally along with pharmaceutically acceptable excipients. The invention also relates to use of hydroxychavicol and/or its derivatives for treating hyperpigmentation.

This application is a non-provisional filing of provisional U.S. patent application No. 60/250,106 filed on Oct. 9, 2009.

### FIELD OF THE INVENTION

The present invention relates to treatment of CNS disorders/diseases by the use of hydroxychavicol and/or its derivatives. More specifically, it relates to the use of hydroxychavicol for treating Alzheimer's disease, a composition comprising hydroxychavicol and/or its derivatives and a process for its preparation.

The present invention also relates to use of hydroxychavicol and/or its derivatives for treating hyperpigmentation.

### BACKGROUND AND PRIOR ART

Central nervous system (CNS) forms majority of the nervous system and consists of the brain, spinal cord, as well as retina. It integrates the information received from, and coordinates the activity of all parts of the body, a fundamental role in the control of behavior together with the peripheral nervous system.

A disorder or a disease in the central nervous system affects either the spinal cord or brain. There are many central nervous system diseases such as Alzheimer's disease and amyotrophic lateral sclerosis (neurodegenerative), multiple sclerosis (autoimmune and inflammatory), Krabbe's disease, Huntington's disease (genetic) etc.

Alzheimer's disease (AD) is one of the most common forms of neurodegenerative disease reported to have affected more than 35 million people worldwide. Also called as Senile Dementia of the Alzheimer Type (SDAT), it is characterized by memory loss in the early stage such as difficulty in remembering recently learned facts. Later, the symptoms advances to confusion, rapid change in mood, language breakdown, long term memory loss etc. Over the time, the individual suffers from loss of bodily functions, ultimately leading to death.

Though the cause is not well understood, the disease is associated with formation of plaques and tangles due to accumulation of abnormal levels of Aβ and tau proteins in the brain. Hence, the disease is also called as proteopathy or taupathy. In addition, the disease is also characterized by reduction in the presence of acetylcholine, a neurotransmitter essential for transmitting signals between neurons.

Aβ proteins or β-amyloids having length of about 39-43 amino acids are a fragment of amyloid precursor protein (APP). APP is a transmembrane protein that penetrates through the neuron's membrane and is crucial for neurons growth, survival and post-injury repair. In Alzheimer's disease, β-amyloids are formed by cleavage of APP through proteolysis and are deposited outside neurons in dense formations known as senile plaques.

The Aβ region of amyloid precursor protein (APP) is cleaved by three types of proteases, which are designated as α-, β- and γ-secretases. Processing by β- and γ-secretases cleaves on the N- and C-terminal ends of the Aβ region respectively, releasing Aβ, whereas α-secretase cleaves within the Aβ sequence (Mills and Reiner, 1999). γ-Secretase cleaves at several adjacent sites to yield Aβ species containing 39-43 amino acid residues. A substantial body of evidence indicates that accumulation of Aβ in the brain, particularly longer species containing 42 or 43 residues (long Aβ), is an important step in the pathogenesis of AD (Small and McLean, 1999).

Tau proteins, also called as microtubule-associated proteins stabilize microtubules upon phosphorylation. Forming one of the components of cytoskeleton, microtubules play a role in cellular processes such as mitosis, cytokinesis and vesicular transport apart from serving as structural components within the cells. In Alzheimer's disease, tau pairs with other threads due to hyperphosphorylation creating neurofibrillary tangles and disintegrating neuron's transport system.

Plaques and tangles are believed to disturb the activities of nerve cells by blocking the communication amongst them. In addition, they are found to provide obvious stimuli for inflammation which is suggested to significantly contribute to pathogenesis of the disease (Neurobiology Aging. 2000 May-June; 21(3):383-421). Also, excess tumor necrosis factor-alpha (TNF-alpha) is centrally involved in the pathogenesis of Alzheimer's disease (Journal of Neuroinflammation 2008, 5:2).

Inflammatory components related to AD include microglia and astrocytes (Kalaria, 1999; D'Andrea et al., 2004). Upon inflammatory stimulation, astrocytes proliferate and produce diverse intercellular mediators such as NO (Nitric Oxide) and TNF-α (Galea et al., 1992; Sawada et al., 1989; Simmons and Murphy, 1992). Exposure of microglia to Aβ causes its activation leading to an increase in cell-surface expression of MHC II along with increased secretion of pro-inflammatory cytokines interleukin-1beta (IL-1β), interleukin-6 (IL-6) and tumor necrosis factor alpha (TNF-α), macrophage inflammatory protein-1 alpha (MIP-1α) and monocyte chemo-attractant protein-1 (Rogers and Lue, 2001).

NO produced upon inflammatory stimulation causes neuron death. For example, studies determined that blocking NOS in the brain impedes cell death that occurs from stroke as well as in other disorders such as Parkinson's disease (Togo et al., 530 2004).

As a result of inflammation, normal brain molecules are disrupted and this can cause amyloid-beta proteins in the brain to misfold and these are thought to have a critical role in the development of AD. Thus, drugs that regulate the production of Aβ by inhibiting or modulating secretase activity could provide effective therapeutics for AD.

Several compounds/drugs like donepezil, memantine etc have been prescribed for treating Alzheimer's disease.

Donepezil acts as acetylcholinesterase inhibitor thereby increasing the concentration of acetylcholine in the brain essential for processing memory and learning. Other drugs such as Rivastigmine and Galantamine are also used as acetylcholinesterase inhibitors.

Memantine is prescribed for advanced symptoms of AD and it acts as NMDA receptor antagonist. NMDA receptor is the receptor to which N-methyl D-aspartate binds and is a glutamate receptor involved in controlling synaptic plasticity and memory function. In Alzheimer's disease, the NMDA receptor is overstimulated by the excess amounts of glutamate in the brain causing the death of cells. This process is known as excitotoxicity. By blocking the receptor, memantine inhibits overstimulation by glutamate and prevents excitotoxicity.

Though acetylcholinesterase inhibitors and NMDA receptor antagonist are known to be used, none of them halt the progression of disease and hence, there is no cure for the disease. Despite no cure, still there exists a need for compounds/drugs which can delay the onset of disease or the compounds/drugs which can prevent/manage the disease.

Hydroxychavicol is a compound which can be either extracted from plants or can be synthesized. It is one of the main components in members of the plant piperaceae. The compound is known to have many roles in the art such as suppression of COX-1/COX-2 enzyme activity (Br J Pharmacol. 2007 September; 152(1):73-82. Epub 2007 Jul. 16), prevention and treatment of oral infections (Antimicrob. Agents Chemother. January 2009, p. 216-222, Vol. 53, No. 1) etc.

U.S. Pat. No. 7,252,845 disclose a pharmaceutical, a cosmetic or a dietary supplement comprising: a) 50.0-99.5% (w/w) 1′-acetoxychavicol acetate; b) 0.5-98% (w/w) of one or more compounds selected from the group consisting of 1′-acetoxyeugenol acetate, trans-p-coumaryl diacetate, coniferyl diacetate, 1′-hydroxychavicol acetate, 1′-hydroxychavicol, p-hydroxy-trans-cinnamaldehyde, p-methoxy-trans-cinnamylalcohol and 3,4-dimethoxy-trans-cirmamylalcohol for the treatment of IgE mediated allergic conditions.

WO/2001/66097 talks about the use of hydroxychavicol as an antimicrobial active substance against Pseudomonas.

WO/2003/082233 discloses the use of allyl-phenol compounds such as hydroxychavicol for treating male-pattern alopecia, acne, seborrhea and dandruff.

The present invention proposes new use of the compound hydroxychavicol and/or its derivatives along with a composition comprising the compound(s) and a process thereof.

### SUMMARY OF THE INVENTION

The present invention relates to a method of treating CNS disorders/diseases by use of hydroxychavicol and/or its derivatives. Particularly, the invention relates to the use of hydroxychavicol for treating Alzheimer's disease, a composition comprising hydroxychavicol and/or its derivatives and a process for its preparation.

Hydroxychavicol modulates the activity of β and γ-secretases which are responsible for producing β-amyloid protein which is a major cause of Alzheimer's. In addition, the compound regulates/modulates the activity of inflammatory components and other markers stimulated by the β-amyloid deposition, thus controlling the overall immune system.

The present invention also relates to use of hydroxychavicol and/or its derivatives for treating hyperpigmentation.

The compound and/or its derivatives can be included in various compositions suitable for intravenous, intramuscular, topical, local, intraperitoneal or other forms of administration.

### BRIEF DESCRIPTION OF ACCOMPANYING FIGURES

FIG. 1: Structure of Hydroxychavicol (HC).

FIG. 2: HPLC chromatogram of Hydroxychavicol (HC).

FIG. 3: The effect of HC treatment (0.5, 1, 2 and 4 mg/kg/p.o.) on passive avoidance performance after ICV injection of STZ in rats as indicated by initial and retention latencies. Student's ‘t’ test. Values are expressed as mean±S.E. *p<0.01, drug treated group compared to STZ Control group.

FIG. 4: The graph represents the dose dependent effect of HC on the expressions of TNF-α, IL-1β and IL-6 in supernatant from brain tissue homogenate in ICV STZ treated rats. Student's ‘t’ test. Values are expressed as mean±S.E. *p<0.001, drug treated group compared to STZ Control group.

FIG. 5: The line graph represents the dose dependent effect of HC on the expression of NO in supernatant from brain tissue homogenate in ICV STZ treated rats. Student's ‘t’ test. Values are expressed as mean±S.E. *p<0.001, drug treated group compared to STZ Control group.

FIG. 6: Comparison of acetylcholinesterase levels in Sham Control, CSF Control, STZ Control and HC-treated groups. Values are expressed as mean±S.E. *p<0.01, **p<0.001, drug treated group compared to STZ Control group.

FIG. 7: The graph represents the dose dependent effect of HC on the expression of β-secretase in supernatant from brain tissue homogenate in ICV STZ treated rats. Student's T test. Values are expressed as mean±S.E. **p<0.001, drug treated group compared to STZ Control group.

FIG. 8: The graph represents the dose dependent effect of HC on the expression of γ-secretase in supernatant from brain tissue homogenate in ICV STZ treated rats. Student's ‘t’ test. Values are expressed as mean±S.E. *p<0.01, **p<0.001 drug treated group compared to STZ Control group.

FIG. 9: The quadrant plot represents the dose dependent effect of HC on the expressions of CD3+ and CD19+ cell population in peripheral blood lymphocytes from ICV STZ treated rats. Values are expressed as mean±S.E.

FIG. 10: The histogram plot represents the dose dependent effect of HC on the expression of IFN-γ in peripheral blood lymphocytes from ICV STZ treated rats. Values are expressed as mean±S.E

### DETAILED DESCRIPTION OF THE INVENTION

The present invention relates to a method of treating CNS disorders, said method comprising step of administering to a subject in need thereof a therapeutically effective amount of hydroxychavicol and/or its derivatives or a composition comprising hydroxychavicol and/or its derivatives optionally along with pharmaceutically acceptable excipients.

In another embodiment of the present invention, the disorders are selected from a group comprising Alzheimer's disease, Parkinson's disease, Huntington's disease and Multiple sclerosis.

In another embodiment of the present invention, the disorder is Alzheimer's disease.

In yet another embodiment of the present invention, the hydroxychavicol and/or its derivatives modulates the activity of secretases.

In still another embodiment of the present invention, the secretases are β-secretase and γ-secretase.

In still another embodiment of the present invention, the hydroxychavicol and/or its derivatives modulates pro-inflammatory cytokines, nitric oxide, malondialdehyde, and cell-surface markers.

In still another embodiment of the present invention, the pro-inflammatory cytokines are TNF-α, IL-1β and IL-6 and IFN-γ.

In still another embodiment of the present invention, the cell-surface markers are CD3 and CD-19.

In still another embodiment of the present invention, the hydroxychavicol and/or its derivatives modulate acetylcholinesterase and glutathione.

In still another embodiment of the present invention, the subject is human being.

The present invention also relates to a method of modulating β-amyloid protein, said method comprising step of exposing the tissue or cells synthesizing secretases with hydroxychavicol and/or its derivatives or with a composition comprising hydroxychavicol and/or its derivatives optionally along with pharmaceutically acceptable excipients.

In still another embodiment of the present invention, the secretases are β-secretase and γ-secretase.

The present invention also relates to a composition comprising hydroxychavicol and/or its derivatives optionally along with pharmaceutically acceptable excipients.

In still another embodiment of the present invention, the composition further comprises compounds used for treating CNS disorders.

In still another embodiment of the present invention, the disorders are selected from a group comprising Alzheimer's disease, Parkinson's disease, Huntington's disease and Multiple sclerosis.

In another embodiment of the present invention, the disorder is Alzheimer's disease.

In still another embodiment of the present invention, the pharmaceutically acceptable excipients are selected from a group comprising antiadherents, binding agents, coating agents, disintegrating agents, fillers and diluents, flavoring agents, colorants, glidants, lubricants, preservatives, sorbents, sweeteners and combinations thereof.

In still another embodiment of the present invention, the composition is formulated into dosage forms selected from a group comprising liquid, troches, lozenges, powder, granule, capsule, tablet, patch, gel, emulsion, cream, lotion, dentifrice, drop, suspension, syrups, elixirs, phyotceuticals and neutraceuticals.

The present invention also relates to a process for preparing a composition comprising hydroxychavicol and/or its derivatives optionally along with pharmaceutically acceptable excipients, said process comprising step of obtaining hydroxychavicol and/or its derivatives and preparing the composition.

In still another embodiment of the present invention, the hydroxychavicol is extracted from plant or is synthesized.

The present invention also relates to a method of treating hyperpigmentation, said method comprising step of topically applying to the skin of a subject in need thereof an effective amount of hydroxychavicol and/or its derivatives or a composition comprising hydroxychavicol and/or its derivatives optionally along with cosmetically or pharmaceutically acceptable excipients.

In still another embodiment of the present invention, the subject is a human

In still another embodiment of the present invention, the composition further comprise one or more skin whitening agents different from hydroxychavicol and/or its derivatives.

The present invention relates to use of hydroxychavicol and/or its derivatives in the treatment of CNS disorders, particularly Alzheimer's disease. The compound modulates the activity of β- and γ-secretases necessary for production of β-amyloid protein which is a major cause of the disease. The compound also modulates the activity of components triggered by β-amyloid deposition.

Hydroxychavicol (HC) is one of the main components present in the members of piperaceae. In the present invention, the compound has been extracted from betel leaf and tested for its activity. Nonetheless, the compound can also be synthesized for its use in the present invention.

The present invention focuses on a method of treating Alzheimer's disease by administering hydroxychavicol to a subject in need thereof. The “subject” means the person having the disease.

Several derivatives can be arrived from hydroxychavicol and these can also be used for treating Alzheimer's disease.

The compound hydroxychavicol and/or its derivatives can be included in various compositions suitable for intravenous, intramuscular, topical, local, intraperitoneal or other forms of administration.

The composition containing hydroxychavicol and/or its derivatives can be formulated into dosage forms selected from a group comprising liquid, troches, lozenges, powder, granule, capsule, tablet, patch, gel, emulsion, cream, lotion, dentifrice, drop, suspension, syrups, elixirs, phyotceuticals and neutraceuticals. The composition may also include other compounds/drugs which are used for treating CNS disorders, particularly Alzheimer's disease.

The present study was undertaken on streptozotocin-induced cognitive dysfunction and associated inflammatory and oxidative damage which is a commonly used experimental model of dementia. The ICV STZ model has been described as an appropriate animal model for sporadic Alzheimer type dementia (Lannert and Hoyer, 1998) and is characterized by a progressive deterioration of memory, cerebral glucose and energy metabolism and presence of oxidative stress (Lannert and Hoyer, 1998; Sharma and Gupta, 2001).

Sub-diabetogenic dose of STZ causes prolonged impairment of brain glucose and energy metabolism leading to impairment in learning and memory, neuro-inflammation and free radical generation (Blokland and Jolles, 1993; Lannert and Hoyer, 1998; Sharma and Gupta, 2001). Upon inflammatory stimulation, astrocytes proliferate and produce diverse intercellular mediators such as nitric oxide (NO) and tumor necrosis factor (TNF-α) thus causing neuronal cell damage (Johnstone et al., 1999; Smits et al., 2002) associated with a markedly increased production of free radicals as indicated with increased malondialdehyde (MDA) (a marker of lipid peroxidation) and depletion of reduced glutathione (an endogenous antioxidant) levels leading to oxidative stress (Veerendra and Gupta, 2003).

On analysis, reduction in the activity of the components responsible for pathogenesis of Alzheimer's disease was observed in the models treated with hydroxychavicol. Reduction in the activity of components responsible for the cause of other CNS disorders was also observed and hence the compound and/or its derivatives may also be used in treating CNS disorders like Parkinson's disease, Huntington's disease, multiple sclerosis etc.

The invention is elaborated with the help of following examples. However, these should not be construed to limit the scope of invention.

### Example 1

### Extraction and Isolation of HC from the Leaves of Piper Betel

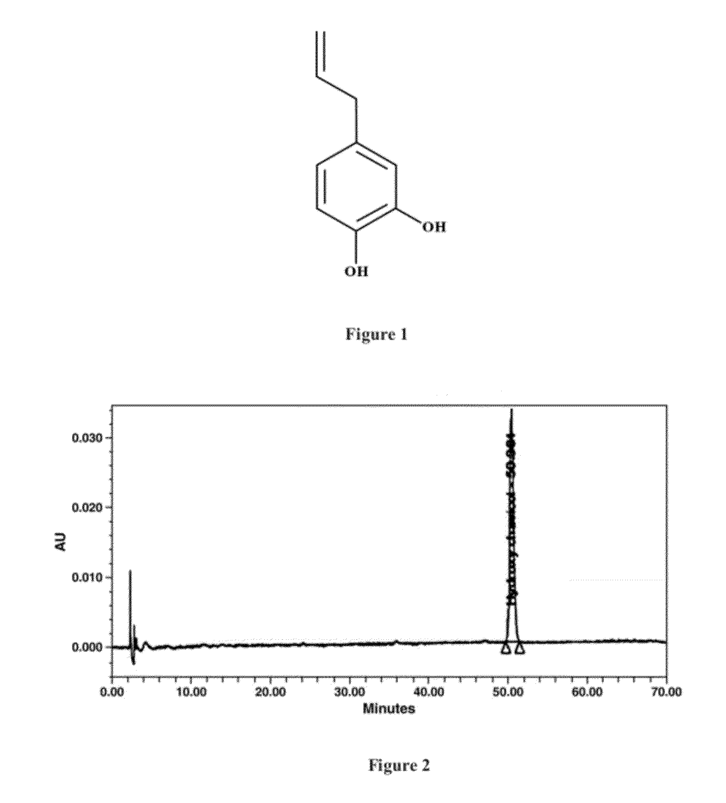

Freshly procured leaves of Piper betel (1 kg) were extracted in boiling water (3 l) with stirring for 4 h. The resulting extract was filtered through muslin cloth, centrifuged, and concentrated to one-sixth of the original volume under reduced pressure at temperature of 50±5° C. on a film evaporator. The concentrated volume was fractionated with chloroform and the chloroform fraction was concentrated under reduced pressure to yield a residue (5.06 g) containing 80% hydroxychavicol (HC), as monitored by high-pressure liquid chromatography (HPLC) and thin-layer chromatography. The Hydroxychavicol enriched residue (5.0 g) was chromatographed on a silica gel column (200 g; 100 to 200 mesh filter; 60 cmby 3.2 cm (Loba-Chemie, India) using 1.0% methanol in chloroform (vol/vol) as eluting solvent. Fractions of 100 ml each were collected and subjected to thin-layer chromatography in CHCl3-MeOH (19:1). The fractions containing pure HC were pooled, and the desired compound (FIG. 1) was crystallized from benzenepetroleum ether as a colourless solid (2.56 g), mp 48° C. (Chang et al., 2002). HC was characterized by spectral analysis. The purity of this compound and its concentration in the crude and chloroform extracts were established by HPLC (FIG. 2).

Hydroxychavicol used in the present invention is interchangeably referred as drug or compound or test material in the description.

### Example 2

### Quantification

HC exhibited a linear response in the concentration range of 17.5 μg/ml to 35 μg/ml, and the calibration curve was prepared by using the multipoint calibration curve method. A working solution was injected in different concentrations. An excellent calibration curve was obtained for hydroxychavicol (r 190 2=0.998886) determined on the basis of six levels of concentration.

### Example 3

### Animals

Adult male Wistar rats, 20-24 weeks old weighing 320-360 g and Swiss albino mice, 10-12 weeks old weighing 24-28 g at the start of the experiment were housed in a temperature-controlled colony room under light/dark cycle. These were given free access to pellet food and water throughout the experiment. All the behavioural experiments were carried out between 11 a.m. and 4 p.m.

### Example 4

### Acute Oral Safety Study

Three female Balb/C mice, fasted 3-4 h prior to the test, were used for each step and observed individually after dosing at least once during the first 30 min, and periodically during the first 24 h, with special attention given during the first 4 h, and daily thereafter, for a total of 14 days. Simultaneously, general behaviour and any toxic symptoms produced by the test material were observed for 14 days for routine pharmacological parameters such as cyanosis, tremors, convulsions, ataxia, body tone, muscle tone, piloerection, salivation, tail flick, drowsiness, alertness, spontaneity, diarrheoa, pupil size, ptosis, breathing rate, urination etc.

No untoward symptom or any mortality was observed in mice treated with HC up to a maximum oral dose of 1000 mg/kg with no change in general behaviour when compared to the normal Control group.

### Example 5

### Experimental Procedure

Rats were divided into the following groups of six animals per group. These groups were: 1. Sham-operated group (Sham Control), 2. CSF Control group (CSF Control) that received bilateral ICV injection of artificial CSF (ACSF) (10 μl on each side) as the solvent of STZ 3. STZ-injected group (STZ Control) which received ICV injection of STZ (10 μl on each side) and Groups 5, 6, 7 and 8 were the drug treated groups receiving 0.5, 1, 2 and 4 mg/kg of HC from day 0 to day 21 after the surgery. Drugs for oral administration were freshly prepared as a suspension of HC in doses of 0.5, 1, 2 and 4 mg/kg in 1% w/v acacia gum and administered orally to rats once daily for the duration of the experiment. The above said doses were taken up for the study because initially in-vivo TNF-α were estimated with a broad range of dose levels of HC and the range of oral doses that showed optimum effect were taken up for the study.

### Example 6

### Intracerebroventricular Injection of Streptozotocin

The rats were anesthetized and placed in a Stoelting stereotaxic apparatus (Ugo Basile, Italy) (incisor bar −3.3 mm, ear bars positioned symmetrically). The scalp was cleaned with iodine solution, incised on the midline and a burr hole was drilled through the skull 0.8 mm posterior to bregma, 1.4 mm lateral to saggital suture, and 3.4 mm beneath the surface of brain, according to the stereotaxic atlas (Paxinos and Watson, 1986). STZ and HC-treated STZ groups were given a bilateral ICV injection of freshly dissolved STZ (3 mg/kg) in cold artificial CSF at a volume of 10 μl on each side. The injection was repeated on day 3. In the CSF Control group, only artificial CSF (120 mM NaCl; 3 mM KCl; 1.15 mM CaCl2; 0.8 mM MgCl2; 27 mM NaHCO3; and 0.33 mM NaH2PO4 adjusted to pH 7.2) was ICV injected. Post-operatively, special care was undertaken until spontaneous feeding was restored.

### Example 7

### Single Trial Passive Avoidance Test

Memory retention deficit was evaluated by a step through passive avoidance apparatus according to the method previously described by Mojard et al. (2007) on days 19th and 20th after 1st injection of STZ. On the acquisition trial, each rat was placed in the lighted chamber and after 60 s of habituation period, the guillotine door separating the lighted and dark chamber was opened, and the initial latency (IL) to enter the dark chamber was recorded. Immediately after the rat entered the dark chamber, the guillotine door was closed and an electric foot shock (75 V, 0.2 mA, 50 Hz) was delivered to the floor grids for 3 s. Five seconds later, the rat was removed from the dark chamber and returned to its home cage. Twenty-four hours later, the retention latency (RL) time was measured in the same way as in the acquisition trial, but foot shock was not delivered, and the latency time was recorded to a maximum of 600 s.

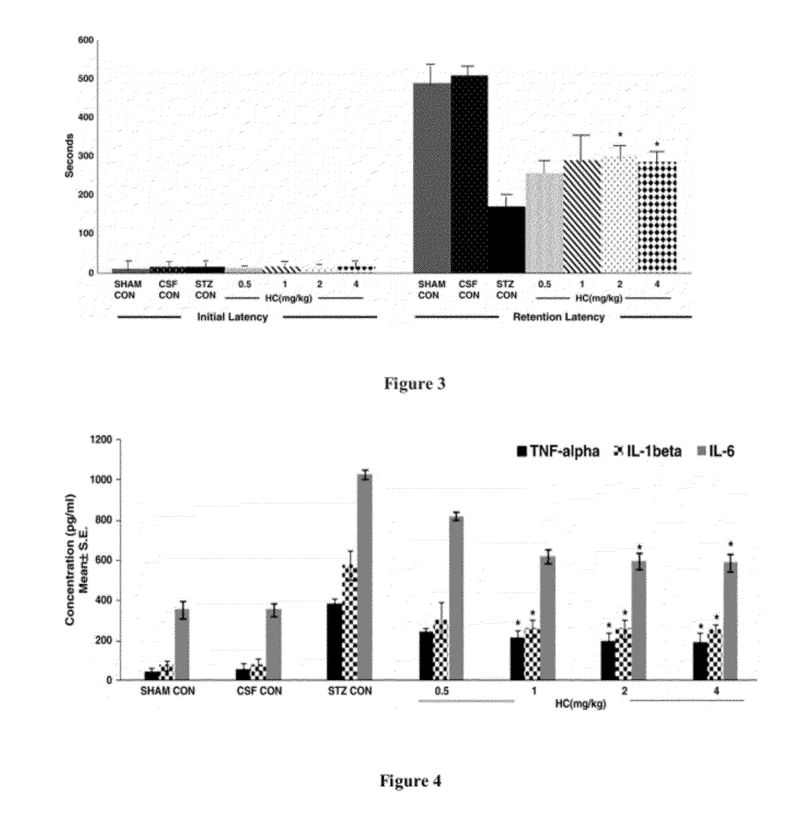

The results from the passive avoidance test show that vehicle-treated ICV STZ rats showed impairment of learning and memory as evidenced by significantly reduced retention latencies. The mean initial latency on day 19 did not differ significantly between the sham, vehicle-treated, ICV STZ group and HC 0.5, 1, 2 and 4 mg/kg treated ICV STZ group. The initial latency was 11.6±2.12 s, 18.13±2.6 s, 15.07±2.09 s, 12.21±1.42 s, 17.33±2.6 s, 15.92±1.09 s and 18.21±2.96 s. On day 20, the mean retention latency in ICV STZ group was significantly less (173±28 s) as compared to that of sham rats (492±31.22 s). The group that was treated with HC, both 1 and 2 mg/kg p.o., showed significant reversal of transfer latency. The mean retention latency was 291.87±25.66 s and 301.09±13.67 s, respectively, which was significantly higher than STZ Control group indicating improved acquisition or retention of memory (FIG. 3). The improvement in passive avoidance behaviour shown by improved acquisition and/or retention of memory indicates an increased capacity to learn in rats treated with HC.

### Example 8

### Tissue Preparation

On day 21, blood was collected from the retro-orbital plexus of the experimental animals for cell-surface marker study and cytokine estimations. The brain of the animals was removed and rinsed with ice-cold isotonic saline. 4 ml/g tissue of extraction buffer containing 1 mM phenylmethylsulfonyl fluoride, 1 mg/ml aprotinin and 0.05% Tween 20 in phosphate buffered saline were added to the tissues. Tissues were homogenized on ice with a polytron and homogenate was centrifuged at 5000 g for 15 min. Aliquots of the supernatant were separated and used for biochemical analysis. Supernatants were stored at −80° C. until cytokine analysis (Magari et al., 2004).

### Example 9

### Estimation of β-Secretase and γ-Secretase

Almost all of the work on APP secretases has been based on the fact that inhibition of Aβ (especially Aβ42) production will block or even reverse the cognitive decline in AD. A decrease in the activity of β- and γ-secretase implies decrease in the concentration of Aβ and thereby a protective effect against the disease.

The tissue samples from all the experimental groups were tested for β- and γ-secretase activities by the addition of a secretase-specific peptide conjugated to the reporter molecule EDANS (5-((2-Aminoethyl)amino) naphthalene-1-sulfonic acid) and DABCYL 4-(4-dimethylaminophenyl)diazenylbenzoic acid. In the uncleaved form, the fluorescent emissions from EDANS are quenched by the physical proximity of the DABCYL moiety which exhibits maximal absorption at the same wavelength (495 nm). Cleavage of the peptide by the secretase physically separates the EDANS and DABCYL allowing the release of a fluorescent signal. Both β- and γ-secretases were estimated using commercially available kits based on sandwich and competitive ELISA technique (R&D Systems, MN, USA) according to the manufacturers' instructions.

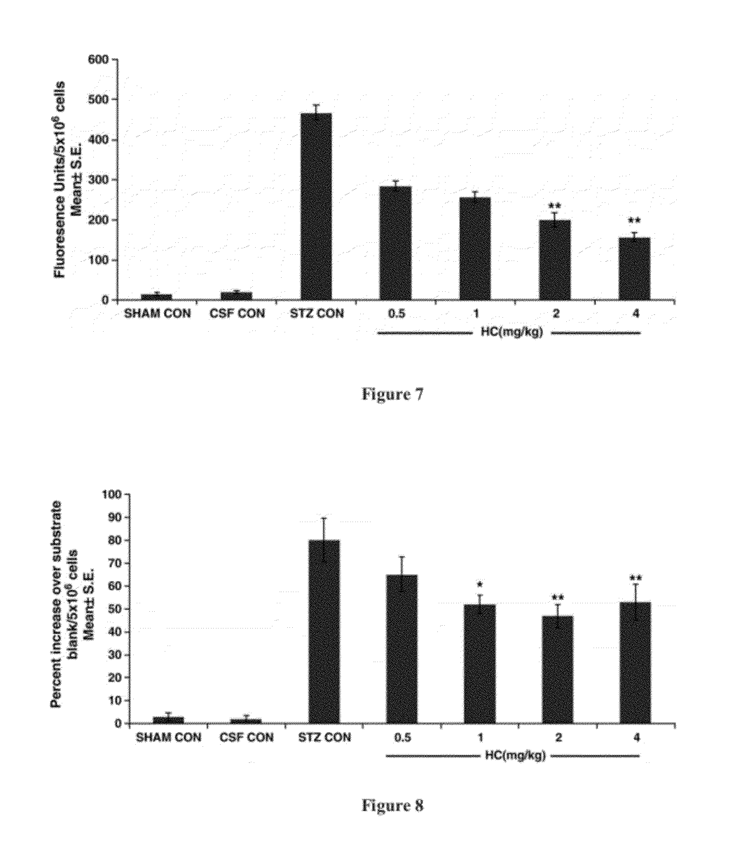

Exposure to chronic oral treatment of HC at graded doses of 0.5, 1, 2 and 4 mg/kg resulted in the decreased concentration of both β- and γ-secretases. The level of secretase enzymatic activity in the tissue preparation of the experimental groups was proportional to the fluorometric reaction (FIGS. 7 and 8). Treatment of HC in graded oral doses for 21 days resulting in decreased enzymatic activity of both β- and γ-secretase activities, shows protective effect of the compound.

The protective effect of hydroxychavicol in decreasing β- and γ-secretase activities proves that the compound is useful in treating Alzheimer's disease. In order to further establish the role of compound, its effect on the inflammatory markers and other components linked to Aβ deposition was analysed. If the compound has decreased the activity of β- and γ-secretase, then it should have the effect on other factors stimulated by Aβ deposition. To establish this and to further confirm the compound's role in treating the disease, we have analysed the effect of compound on other factors playing a role in pathogenesis of the disease.

### Example 10

### Quantification of IL-1β, TNF-alpha, IL-6 and NO in Supernatant from Tissue Homogenate

Samples on day 21 from different groups of animals were prepared for the analysis of cytokines and mediators as described above. IL-1β, TNF-α, IL-6 and NO were estimated using commercially available kits based on sandwich and competitive ELISA technique All cytokine concentrations were carried out by means of colorimetric measurement at 450 nm on an ELISA plate reader by interpolation from a standard curve (Magari et al., 2004).

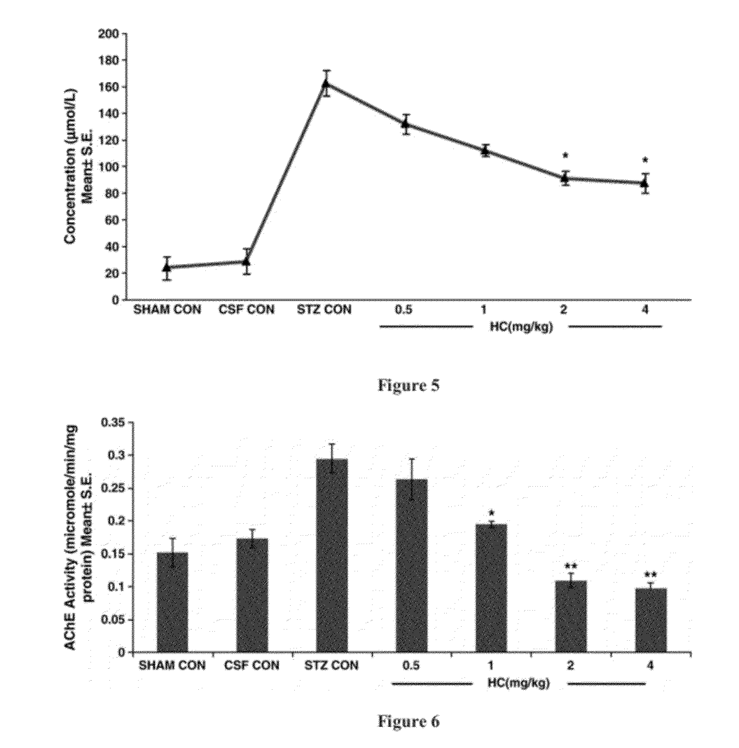

HC at graded doses of 0.5, 1, 2 and 4 mg/kg significantly decreased the TNF-α, IL-1β, IL-6 and NO (FIGS. 4 and 5) levels in a dose dependent manner. Significant inhibition of TNF-α, IL-1β, IL-6 and NO parameters was observed at higher dose levels of 2 and 4 mg/kg per oral.

### Example 11

### Measurement of Lipid Peroxidation

Malondialdehyde (MDA) formed from the breakdown of polyunsaturated fatty acids serves as a convenient index for determining the extent of the peroxidation reaction that reacts with thiobarbituric acid (TBA) to give a red species absorbing at 535 nm. Lipid peroxidation was estimated by the method of Buege and Aust (1978). Phosphate buffer (0.9 ml) was mixed with the tissue homogenate (10%, 0.1 ml) and TBA, TCA, HCl solution (2 ml) was added. After the solution was incubated at 100° C. for 15 min, the tubes were cooled and then centrifuged at 3000 rpm for 10 min. Optical density was measured against blank at 535 nm.

Intracerebroventricular administration of artificial CSF had no effect on brain MDA levels when compared to the sham-operated groups. Central STZ administration caused a marked increase in free radical generation and a significant rise in brain MDA levels when these were compared with the CSF control rats. However, treatment of HC (0.5, 1, 2 and 4 mg/kg, p.o.) significantly prevented the increase in MDA levels with a marked protective effect being observed at the highest dose level of 4 mg/kg. ICV STZ increased the MDA concentration from 132.56±9.23 nmol/mg in Sham Control group to 415.21±10.07 nmol/mg in STZ Control group. Oral administration of HC at 2 and 4 mg/kg decreased the MDA levels to 237.19±3.44 nmol/mg and 220.68±4.32 nmol/mg respectively (Table 1).

### Example 12

### Estimation of Reduced Glutathione

Glutathione an essential tripeptide is an antioxidant found in all animal cells. It reacts with the free radicals and can protect cells from singlet oxygen, hydroxyl radical and superoxide radical damage.

Reduced glutathione (GSH) in the brain was estimated according to the method described by Ellman (1959). 1 ml supernatant was precipitated with 1 ml of 4% sulfosalicylic acid and cold digested at 4° C. for 1 h. The samples were centrifuged at 1200×g for 15 min at 4° C. To 1 ml of this supernatant, 2.7 ml of phosphate buffer (0.1 M, pH 8) and 0.2 ml of 5,5′-dithiobis-(2-nitrobenzoic acid) (DTNB) were added. The yellow colour developed was read immediately at 412 nm using spectrophotometer. The results were calculated using the molar extinction coefficient of chromophore (1.36×104 M−1 cm−1) and expressed as a percentage of the control.

The ICV STZ injection showed a significant decline in the brain GSH activity compared to artificial CSF-injected rats. However, oral administration of HC (0.5, 1, 2 and 4 mg/kg), significantly ameliorated the reduction in GSH activity compared to STZ-injected group. The higher doses of HC (2 and 4 mg/kg, p.o.) treatment showed a more marked effect in restoring GSH activity compared to lower dose. The brain GSH levels were depleted from 419.62±8.11 μg/g in Sham Control group to 223.21±6.08 μg/g in STZ Control group. The levels of GSH were restored to 371.69±6.17 μg/g and 372.87±3.18 μg/g in HC (2 and 4 mg/kg p.o.) treated groups respectively (Table 1).

TABLE 1
Effect of HC treatment on level of MDA (LP) and
GSH concentrations in supernatant from brain
tissue homogenate in ICV STZ treated rats.

| Treatment | MDA (LP) nmol/mg | GSH (μg/g) |
|---|---|---|
| Sham Control | 132.56 ± 9.23 | 419.62 ± 8.11 |
| CSF Control | 145.17 ± 2.70 | 427.18 ± 6.97 |
| STZ Control | 415.21 ± 10.07 | 223.21 ± 6.08 |
| HC—0.5 mg/kg | 301.15 ± 6.34 | 359.44 ± 5.47 |
| | (27.47↓)* | (61.03↑)* |
| HC—1.0 mg/kg | 252.18 ± 4.08 | 362.78 ± 6.24 |
| | (39.26↓)* | (62.52↑)* |
| HC—2.0 mg/kg | 237.19 ± 3.44 | 371.69 ± 6.117 |
| | (42.87↓)* | (66.52↑)* |
| HC—4.0 mg/kg | 220.68 ± 4.32 | 372.87 ± 3.18 |
| | (46.85↓)* | (67.04↑)* |

Student's ‘t’ test. Values are expressed as mean ± S.E.
*p < 0.001, drug treated group compared to STZ Control group. Values in parenthesis denote the percentage activity against STZ Control group.
↑stimulation in expression;
↓suppression in expression.

### Example 13

### Acetylcholinesterase Assay

AChE activity was estimated according to the method of Ellman et al. (1961) with minor modifications as described by Das et al. (2002) using acetylthiocholine iodide (1 mmol/l) as substrate. A kinetic profile of the enzyme activity was measured at the interval of 15 s at 412 nm by ELISA plate reader. Protein was estimated by the methods of Lowry et al. The specific activity of AChE is expressed in mmol/min/mg of protein.

Intracerebroventricular administration of ACSF had no effect on brain acetylcholinesterase levels compared with the sham-operated rats. In contrast, the ICV STZ injection showed a significant increase in the brain AChE activity compared to the ACSF-injected rats. However, chronic oral administration of HC (0.5, 1, 2 and 4 mg/kg) significantly reversed the increase in AChE activity compared to STZ-injected group. The higher doses of HC (2 and 4 mg/kg, p.o.) treatment showed a more marked effect in regulating AChE activity compared to lower dose (FIG. 6).

### Example 14

### Blood Glucose Estimation

On day 21 from 1st STZ injection, blood was collected from retroorbital plexus of all experimental groups and glucose was measured by Accu-Check Sensor Comfort glucostrips (Roche Diagnostics India Pvt. Ltd.) (Saxena et al., 2007).

There was no significant difference in blood sugar level (mg/dl). The observed value of STZ Control group was 84.38±2.76 and HC-treated groups showed 87.21±3.61, 85.33±2.90, 89.64±5.47, 88.73±4.02 at 0.5, 1, 2 and 4 mg/kg, p.o. dose respectively. Therefore, streptozotocin-injected rats exhibited blood glucose levels similar to the Control group.

### Example 15

### Flow Cytometric Studies

### Evaluation of Cell-Surface Markers

100 μl of blood collected from the retro-orbital plexus of the animals was taken in each tube and FITC labeled anti-rat CD3+ and PE labeled CD19+ monoclonal antibodies were added and mixed gently. CD3 monoclonal antibody that reacts with the CD3 differentiation antigen expressed on MHC class I T cytotoxic cells as well as MHC class II T helper cells, was used for the determination of activated T cell populations in the blood and CD19 monoclonal antibody that reacts with CD19 antigen expressed on B cells was used for the estimation of B cell population. Tubes were incubated in dark for 30 min at room temperature. Subsequently, 2 ml of 1×FACS lyses solution was added at room temperature with gentle mixing and these samples were then spinned at (300-400×g). The supernatant was aspirated and samples were given 3 washings of phosphate buffer saline (pH 7.4). The resulting stained cell pellet was resuspended in 500 μl of phosphate buffer saline and was run on a flow cytometer. Acquisition and the analysis were done directly on flow cytometer using Cell Quest Pro software (BD Biosciences) (Bani et al., 2005, 2006).

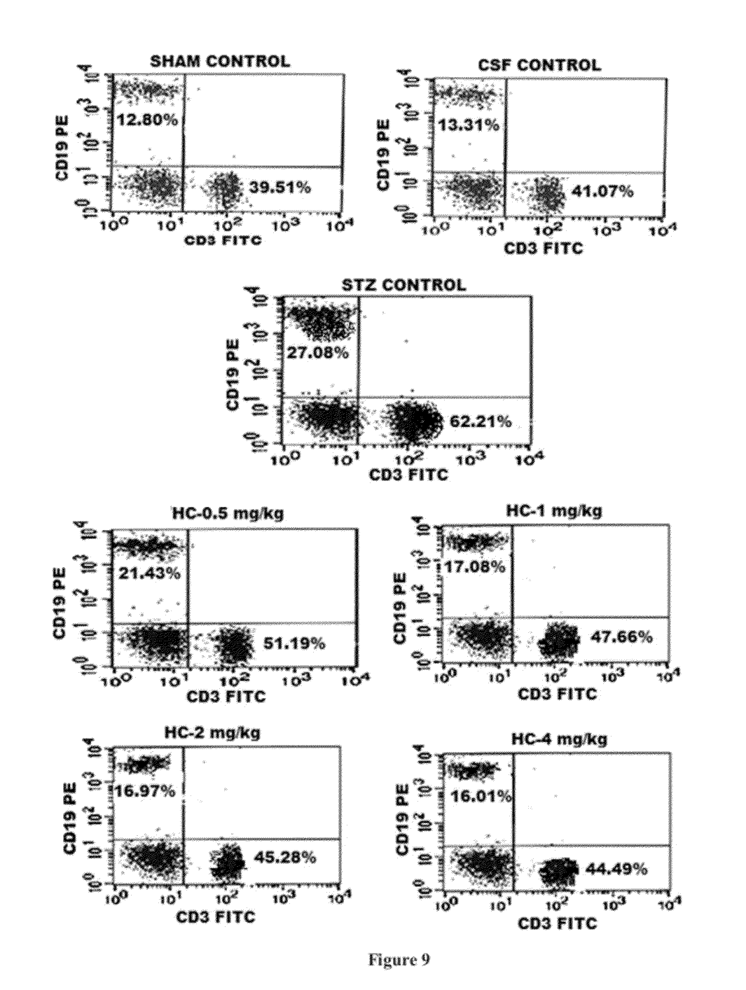

The mean percentage of CD3+ and CD19+ cells in the lymphocyte population was higher in STZ Control group. A significantly higher surface expression of both the markers CD3+(62.21±4.65%) and CD19+(27.08±2.77%) was found in STZ Control group when compared to CD3+(39.51±2.91%) and CD19+(12.80±3.21%) of the sham-operated group. ICV CSF injection did not show any significant change. However, oral administration of HC (0.5, 1, 2 and 4 mg/kg) per oral decreased the overexpressed cell population to (51.19±1.78% CD3+; 21.43±1.39% CD19+), (47.66±1. 43% CD3+; 17.08±2.09% CD19+), (45.28±3.43% CD3+; 16.97±1.88% CD19+) and (44.49±4.55% CD3+; 16.01±1.07% CD19+) respectively (FIG. 9).

### Estimation of Intracellular Cytokines

Magnetic-bead Assisted Cell Sorting (MACS) separated the CD3+ T cells that were then permeabilised with 500 μl of FACS permeabilising solution (Becton Dickinson) for 10 min at room temperature. With this technique, specific cells that have antibody-coated micro beads are separated under a strong magnetic field magnetically from the whole cell suspension according to their cell-surface antigen. After centrifugation, cells were incubated with anti-rat IFN-γ-PE for 30 min in the dark at room temperature. After washing in PBS, cells were fixed in 300 ml of 1% formaldehyde PBS. A total of 10,000-gated events were acquired in on a FACS BD LSR II flow cytometer (Bani et al., 2005, 2006) and analysis was done on flow cytometer using Cell Quest Pro software (BD Biosciences).

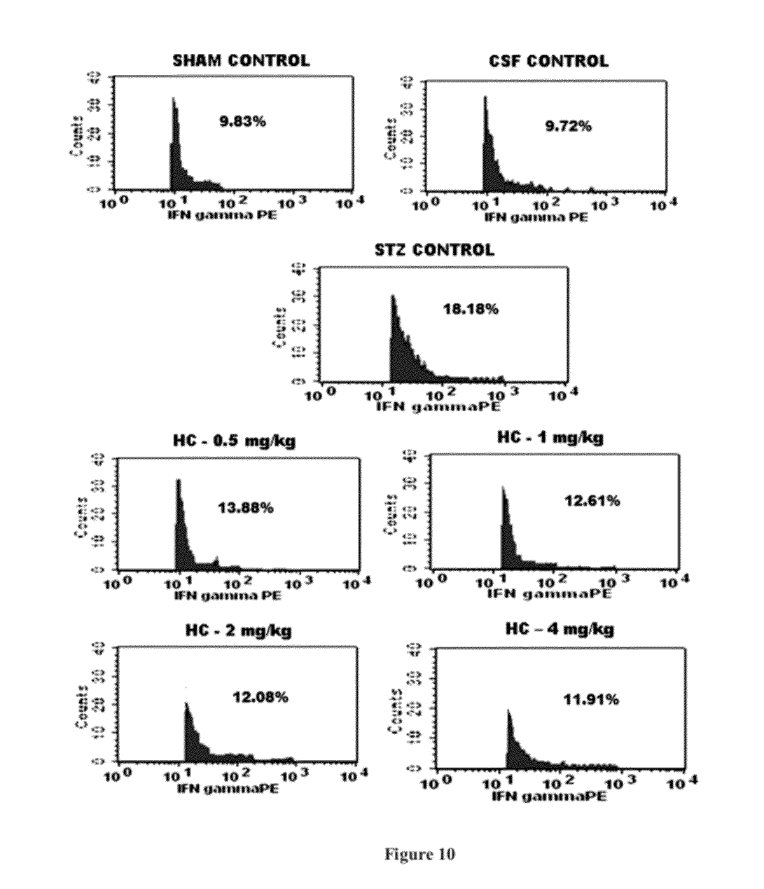

To determine the association of increased expression of pro-inflammatory cytokine IFN-γ with the increased surface markers expression in the lymphocyte population, the IFN-γ was estimated in CD3+ T cells. The Sham Control group showed the expression of IFN-γ to be 9.83±1.19%. This expression increased to 18.18±0.97% in STZ Control group, whereas HC treatment decreased the elevated IFN-γ expression to 12.08±0.21% and 11.91±0.87% at 2 and 4 mg/kg p.o. dose respectively (FIG. 10).

### Example 16

### Comparative Data

In order to prove that the activity of hydroxychavicol is enhanced, it was compared with the crude betel leaf extract, eugenol and isoeugenol invitro with respect to the parameters such as TNF-α, free radicals affecting the disease. The results are as tabulated below in table 2.

TABLE 2
Comparative activity of HC, Crude betel
leaf extract, Eugenol and Isoeugenol.

| | | ROS | | |
|---|---|---|---|---|
| | TNF alpha | Scavenging | ORAC value | DPPH |
| | inhibition | activity | (μmol trolox | inhibition |
| Sample | (IC₅₀ μg/ml) | (IC₅₀ μg/ml) | equivalents/g) | (IC₅₀ μg/ml) |
| Crude | No inhibition | 5 | 696 | 25.2 |
| betel leaf | up to 200 μg/ml | | | |
| extract | | | | |
| Hydroxy- | 88.65 | 0.625 | 29728 | 0.55 |
| chavicol | | | | |
| Eugenol | 114 | 5 | 13921 | 1.5 |
| Isoeugenol | 515 | 0.625 | 15248 | 2.26 |

Note:
Lower the IC50 and higher the ORAC value, better is the activity

It is evident from the above table that hydroxychavicol shows enhanced activity than the crude betel leaf extract, Eugenol and Isoeugenol. TNF-α inhibitory activity and the antioxidant potential shown by the compound is significant in comparison to others.

### Example 17

### Skin Whitening Effect

In addition to the role of hydroxychavicol in the treatment of Alzheimer's disease, it was also found in the present invention that the compound inhibits tyrosinase and Melanin stimulating hormone (MSH) induced melanin. Therefore, the compound is useful for the treatment of hyperpigmentation. The experiments carried to prove the compound's activity against tyrosinase and melanin are as follows:

### Tyrosinase Inhibition:

Pigmentation is a multistep process critically dependent on the functional integrity of tyrosinase, the rate-limiting enzyme in melanin synthesis. Biosynthesis of melanin is initiated by the catalytic oxidation of tyrosine to 3, 4 dihydroxy phenylalanine (dopa) by tyrosinase. Subsequent reactions happen spontaneously eventually resulting in the synthesis of melanin. Under in vitro conditions, tyrosinase enzyme acts on L-Tyrosine forming a pink colored complex. This pink color intensity formed during the reaction is quenched in the presence of the inhibitor.

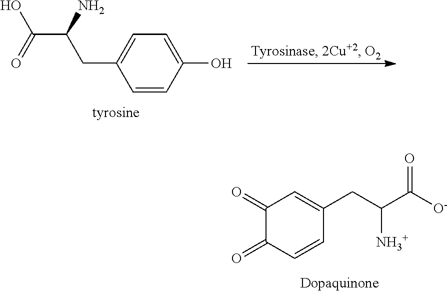

The assay is performed in a 96 well clear microtitre plate. The compound hydroxychavicol, crude betel leaf extract and the reference standard in suitable vehicle (PBS or 0.2% DMSO) is pre incubated with 40 units of Mushroom Tyrosinase enzyme at 37° C. for 10 minutes. The reaction is initiated by adding 0.7 mM L-Tyrosine disodium and the absorbance is read after 10 minutes of incubation at 37° C. in FluostarOptima microplate reader at 492 nm. The dose dependent inhibitory activity of samples is calculated and the results are expressed as IC₅₀ values using Graphpad prism software.

### Inhibition of α-MSH Induced Melanogenesis in B16F1 Mouse Melanoma Cell Line:

Melanin synthesis can be directly studied in live animal cells. B16F1 mouse melanoma cells were seeded in a 6 well microtiter plate at a seeding density of 5000 cells per well in 2 ml DMEM medium per well. After 24 hours of incubation in a CO₂ incubator, melanin production is induced by 0.6 nM μ-MSH by replacing the medium with medium containing μ-MSH. The cells were then treated with the compound hydroxychavicol, crude betel leaf extract and the reference standard over a period of 9 days with renewal of μ-MSH containing medium and sample at regular intervals of 3 days. Control wells were maintained without sample treatment and only with the vehicle used for sample preparation. After the incubation period, the medium was removed and the cells were scraped and washed in PBS. Thereafter, melanin was extracted by 1N NaOH in boiling water bath for 5 minutes. The absorbance of the melanin extract was read at 405 nm in a microplate reader. The inhibitory effect of the samples is calculated based on the decrease of melanin formation. The dose dependent inhibitory activity of samples is calculated and the results are expressed as IC₅₀ values using Graphpad prism software.
The results in the table below show that hydroxychavicol inhibited tyrosinase with an IC₅₀ value of 8 μg/ml and inhibited melanin with an IC₅₀ value of 1.3 μg/ml which is better than the reference standard Ascorbic acid. Though the experiments were conducted with the crude betel leaf extract for comparison, it was found that the crude extract did not show any activity. These results are indicative of the compound's use as a promising skin whitening agent for treating hyperpigmentation.

TABLE 3
Effect of hydroxychavicol on tyrosinase and melanin

| | Tyrosinase | | Inhibition of MSH induced | |
|---|---|---|---|---|
| | inhibition | | Melanin | |
| Hydroxychavicol | IC₅₀ —8 | μg/ml | IC₅₀ —1.3 | μg/ml |
| (90%) | | | | |
| Ascorbic acid | IC50—9.33 | μg/ml | IC50—25 | μg/ml |

1. A method of treating CNS disorders, said method comprising step of administering to a subject in need thereof a therapeutically effective amount of hydroxychavicol and/or its derivatives or a composition comprising hydroxychavicol and/or its derivatives optionally along with pharmaceutically acceptable excipients.

2. The method of claim 1, wherein the disorders are selected from a group comprising Alzheimer's disease, Parkinson's disease, Huntington's disease and Multiple sclerosis.

3. The method of claim 1, wherein the disorder is Alzheimer's disease.

4. The method of claim 1, wherein the hydroxychavicol and/or its derivatives modulates the activity of secretases.

5. The method of claim 4, wherein the secretases are β-secretase and γ-secretase.

6. The method of claim 1, wherein the hydroxychavicol and/or its derivatives modulates pro-inflammatory cytokines, nitric oxide, malondialdehyde, and cell-surface markers.

7. The method of claim 6, wherein the pro-inflammatory cytokines are TNF-α, IL-1β and IL-6 and IFN-γ.

8. The method of claim 6, wherein the cell-surface markers are CD3 and CD-19.

9. The method of claim 1, wherein the hydroxychavicol and/or its derivatives modulates acetylcholinesterase and glutathione.

10. The method as claimed in claim 1, wherein the subject is a human.

11. A method of modulating β-amyloid protein, said method comprising step of exposing the tissue or cells synthesizing secretases to hydroxychavicol and/or its derivatives or with a composition comprising hydroxychavicol and/or its derivatives optionally along with pharmaceutically acceptable excipients.

12. The method of claim 11, wherein the secretases are β-secretase and γ-secretase.

13. A composition comprising hydroxychavicol and/or its derivatives optionally along with pharmaceutically acceptable excipients.

14. The composition of claim 13, wherein the composition further comprise compounds used for treating CNS disorders.

15. The composition of claim 14, wherein the disorders are selected from a group comprising Alzheimer's disease, Parkinson's disease, Huntington's disease and Multiple sclerosis.

16. The composition of claim 14, wherein the disorder is Alzheimer's disease.

17. The composition of claim 13, wherein the pharmaceutically acceptable excipients are selected from a group comprising antiadherents, binding agents, coating agents, disintegrating agents, fillers and diluents, flavoring agents, colorants, glidants, lubricants, preservatives, sorbents, sweeteners and combinations thereof.

18. The composition of claim 13, wherein the composition is formulated into dosage forms selected from a group comprising liquid, troches, lozenges, powder, granule, capsule, tablet, patch, gel, emulsion, cream, lotion, dentifrice, drop, suspension, syrups, elixirs, phyotceuticals and neutraceuticals.

19. A process for preparing a composition comprising hydroxychavicol and/or its derivatives optionally along with pharmaceutically acceptable excipients, said process comprising step of obtaining hydroxychavicol and/or its derivatives and preparing the composition.

20. The process of claim 19, wherein the hydroxychavicol is extracted from plant or is synthesized.

21. A method of treating hyperpigmentation, said method comprising step of topically applying to the skin of a subject in need thereof an effective amount of hydroxychavicol and/or its derivatives or a composition comprising hydroxychavicol and/or its derivatives optionally along with cosmetically or pharmaceutically acceptable excipients.

22. The method of claim 21, wherein the subject is a human.

23. The method of claim 21, wherein the composition further comprises one or more skin whitening agents other than hydroxychavicol and/or its derivatives.
